(19) United States
(12) Patent Application Publication
(10) Pub. No.: US 2015/0361056 A1
(43) Pub. Date: Dec. 17, 2015

(54) CL-20:DNMT COCRYSTAL CRYSTAL STRUCTURE

(71) Applicant: Nalas Engineering Services Inc., Centerbrook, CT (US)

(72) Inventors: Jerry SALAN, Salem, CT (US); David J. am ENDE, East Lyme, CT (US); Stephen R. ANDERSON, Stonington, CT (US)

(73) Assignee: Nalas Engineering Services Inc., Centerbrook, CT (US)

(21) Appl. No.: 14/674,146

(22) Filed: Mar. 31, 2015

Related U.S. Application Data
(60) Provisional application No. 61/972,553, filed on Mar. 31, 2014.

(51) Int. Cl. C07D 249/14; C30B 29/54; C07D 487/22; C30B 7/14

(52) U.S. Cl. CPC C07D 249/14; C30B 7/14; C30B 29/54; C07D 487/22

(57) ABSTRACT

A cocrystal of CL-20 (2,4,6,8,10,12-hexanitro-2,4,6,8,10,12-hexaazaisowurtzitane) and DNMT (1-methyl-3,5-dinitro-1,2,4-triazole) was formed through a resonant acoustic mixing process. The resulting cocrystal comprised an essentially 1:1 stoichiometric ratio between these coformers. The cocrystal advantageously has decreased sensitivity when compared with a pure CL-20 sample, and maintains thermal stability and comparable energetic performance.

### CROSS REFERENCE TO RELATED APPLICATION(S)

This application claims the benefit of U.S. Provisional Patent Application No. 61/972,553, which was filed Mar. 31, 2014. The disclosure of application Ser. No. 61/972,553 is incorporated by reference herein in its entirety.

### UNITED STATES GOVERNMENT RIGHTS

This invention was made with U.S. Government support under contact INIT579 awarded by United States Army ARDEC. The U.S. Government has certain rights in the invention.

### BACKGROUND

The energetic materials community is constantly seeking new materials with improved physical properties and performance. As the synthesis of novel energetic entities is a challenging and tedious endeavor, alternative strategies for producing new materials are of high interest. Energetic materials are frequently cast aside due to poor properties as a formulated ingredient. Amongst many, the properties of interest include density, melting point, decomposition energy, and mechanical properties of the resulting formulation. These factors coupled with the economics of system integration make the selection of ingredients extremely challenging.

Strategies to address the Insensitive Munitions (IM) challenges thus far have employed the use of various chemical and mechanical methods to mitigate the response to stimuli. The energetics community has invested millions of dollars investigating various salts, polymorphs, and amorphous energetic solids. In addition, a large investment in crystallization of various morphology and differing particle size energetic materials have been produced and tested in energetic formulations. These solutions have demonstrated the importance of the chemical and physical form of the energetic material within the formulation. However, the testing is quite limited to the number of energetic materials currently available and no materials have completely solved the IM challenges presented. There are less mature 6.1 technologies, including new synthesis approaches, being pursued to develop new materials for formulations.

Crystal engineering by cocrystallization of coformers is a well-recognized and popular method to prepare new entities from established components. Scientists in fields ranging from the pharmaceutical industry to material sciences have taken advantage of this technology in impressive settings. Through engineering, cocrystals have the potential to display new properties compared to the discrete components. Of interest for energetic formulations, energetic cocrystals can potentially lead to the improvements of formulation density, thermal stability, hygroscopicity, solubility, and response to stimuli. In the realm of energetic compounds, cocrystallization offers an opportunity to manipulate those molecular environments in the entire material and optimistically tune or modify the sensitivity and physical properties of the new entity. The proposed effort does not require chemical synthesis of new materials, but utilizes mature ingredients (6.2 technologies) to produce the cocrystals for use in formulations.

Cocrystals can be thought of simply as a multi-component crystal. More specifically a cocrystal can be understood as a multi-component molecular crystal in a defined stoichiometric ratio whose components are non-covalently bonded via hydrogen bonding or other van der Waals interactions. A cocrystal is a crystalline molecular complex which may include an ionic pair among the different components and by definition includes solvates (hydrates), inclusion compounds, clathrates and solid solutions (Reutzel-Edens, Susan M. Analytical Techniques and Strategies for Salt/Cocrystal Characterization, Fundamental Aspects of Salts and Cocrystals, in Pharmaceutical Salts and Co-crystals, Edited by Johan Wouters and Luc Quere, RSC Drug Discovery Publishing 2012, incorporated herein by reference in its entirety).

Cocrystals are well known to industry, especially in the pharmaceutical industry and the energetics community. Cocrystals are important to the pharmaceutical industry because they provide an opportunity for tuning the physicochemical properties of active pharmaceutical ingredients (APIs). For example, cocrystals may be used to attenuate solubility, dissolution properties, hygroscopicity, mechanical properties, particle size, thermal properties, stability, and enhance bioavailability of a poorly soluble drug or simply improve chemical stability characteristics.

Similar challenges are encountered in both the pharmaceutical industry as well as the energetic community, including scalability. The use of energetic cocrystals in field operations is directly restricted by the challenges associated with their production on large scale. Traditional methods of forming cocrystals largely rely on solution-phase crystallization or grinding-assisted methods. Solution phase methods can be challenging to develop as they are prone to precipitation of the discrete coformers, solvates of the coformers, other polymorphs of the discrete coformers, and potentially disordered solids or undesired adducts. Given the dual solubility constraints of each coformer, a solution cocrystallization process may not be feasible or even possible for certain cocrystals combinations. Depending on the phase equilibria, solvates of either coformer may form instead. Alternatively, grinding-based methods are restricted due to the sensitivity of energetic materials to stimuli.

Homogeneous mixing of multiple components with significantly different properties can be achieved through the application of a low-frequency acoustic field. This mixing system is designed to keep the sample in resonance at 60 Hz with the ability to adjust the mixing intensity to accelerate the material from low intensity, near 0 g acceleration, to high intensity mixing near 100 g acceleration. The intimate coupling of the payload to the mixing container and the continuous adjustment to keep the payload in resonance facilitates the transfer of the potential energy stored in the mixers mechanical system. The propagation of the low frequency wave creates a uniform shear field throughout the mixing vessel. Thus the method is efficient, rapid, and environmentally friendly. Unlike stirred tank vessels, the mixing provided by resonant acoustics is directly scaleable to multi kilogram scale making it scale-transparent; i.e. the same process can be used on gram as well as kg scale.

Sensitivity in energetic materials is a collective phenomenon in which the environment of a decomposing molecule largely defines the chemistry (activation barriers and reaction rates, as well as decomposition routes and dominating mechanisms). Hence, cocrystallization is a potentially transformative venue to manipulate those molecular environments in the entire material and possibly tune (modify) sensitivity in an efficient and controllable way. In some embodiments, as used herein an energetic compound is any material such as an explosive, propellant, or pyrotechnic that can attain a highly energetic state such as by chemical reactions. Cocrystal materials will be utilized in explosive formulations to address IM challenges including shaped charge jet (SCJ), bullet impact (BI), slow cook-off (SCO), fast cook-off (FCO), and sympathetic detonation (SD). The use of cocrystals (e.g. NTO/HMX cocrystal) vs. discrete materials (e.g. NTO and HMX mechanically mixed) changes the physical properties of not just the resulting material but the cocrystal formulation; the resulting formulation contains a new ingredient and not a mixture of two discrete materials.

In the pursuit of new energetic materials with increased performance, groups on the international scene have reported a small number of energetic cocrystals over the past few years. Unfortunately, the materials offering interesting performance properties have also been found to typically possess the same or worse relative sensitivity towards friction, impact and electrostatic discharge (ESD) than one of the individual components. The application of these new materials to fielded systems has however been hampered by three challenges: a) the surprising difficulty associated with the production of cocrystals of electron-deficient coformers lacking the traditional stabilizing interactions, b) the resulting sensitivity of the novel cocrystals produced to stimuli, and c) the scale up of energetic cocrystals to meet the formulation needs.

2,4,6,8,10,12-Hexanitro-2,4,6,8,10,12-hexaazaisowurtzitane (HNIW, aka. CL-20) is the most powerful explosive known and is currently of high interest in the energetic community for the properties it imbues to various formulations matrices. CL-20 possesses IM qualities when formulated using the desired polymorph, particle size, and morphology, making it a prime candidate for a variety of applications. Its outstanding performance has thus led to a number of publications on various polymorphs, solvates and recently cocrystals. However, the energetics community's recent shift towards IM has spurred a wave of research focused on new insensitive explosives possessing a distinct and stable melt phase.

### SUMMARY

In some embodiments, the present disclosure is directed to a cocrystal comprising 2,4,6,8,10,12-hexanitro-2,4,6,8,10,12-hexaazaisowurtzitane and at least one additional energetic material, wherein the sensitivity of the cocrystal is less than the sensitivity of 2,4,6,8,10,12-hexanitro-2,4,6,8,10,12-hexaazaisowurtzitane. In some embodiments, the cocrystal retains favorable thermal stability characteristics. In some embodiments, the at least one additional energetic material is 1-methyl-3,5-dinitro-1,2,4-triazole. In some embodiments, the molar ratio of 2,4,6,8,10,12-hexanitro-2,4,6,8,10,12-hexaazaisowurtzitane to 1-methyl-3,5-dinitro-1,2,4-triazole is approximately 1:1. In some embodiments, the cocrystal is used in an energetic formulation.

In some embodiments, a unit cell of the cocrystal comprises about 4 molecules of 2,4,6,8,10,12-hexanitro-2,4,6,8,10,12-hexaazaisowurtzitane and about 4 molecules of 1-methyl-3,5-dinitro-1,2,4-triazole. In some embodiments, the cocrystal has a crystalline phase density at 95 Kelvin of about 1.8 g/cm³ to about 1.9 g/cm³.

In some embodiments, the cocrystal is produced via a resonant acoustic mixing process. In some embodiments, the method includes the steps of providing a reaction mixture of 2,4,6,8,10,12-hexanitro-2,4,6,8,10,12-hexaazaisowurtzitane, 1-methyl-3,5-dinitro-1,2,4-triazole, and a wet media, and subjecting the reaction mixture to resonant acoustic mixing. In some embodiments, the reaction mixture is subjected to resonant acoustic mixing for a period of at least about 1 hour. In some embodiments, the reaction mixture is mixed at a g-force acceleration of about 80 g. In some embodiments, the wet media is an organic solvent. In some embodiments, the organic solvent is acetonitrile. In some embodiments, cocrystal is produced via a mechanicochemical process selected from the group consisting of: solution crystallization, slurry crystallization, and the like.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A shows the chemical structure of 2,4,6,8,10,12-hexanitro-2,4,6,8,10,12-hexaazaisowurtzitane.

FIG. 1B shows the chemical structure of 1-methyl-3,5-dinitro-1,2,4-triazole.

FIG. 2 shows a graph of the excess enthalpy of the cocrystallization of CL-20 with HMX, TNT, and DNMT.

FIG. 3 portrays the unit cell of a CL-20:DNMT cocrystal consistent with some embodiments of the present disclosure.

FIG. 4 shows a powder x-ray diffraction pattern of the CL-20:DNMT cocrystal shown in FIG. 3 against the diffraction pattern predicted from the single crystal structure.

FIG. 5 shows differential scanning chromatography data for the CL-20:DNMT cocrystal shown in FIG. 3 against the discrete coformers (heating rate of 5° C./min).

FIG. 6 shows a method of making a composite material consistent with some embodiments of the present disclosure.

### DETAILED DESCRIPTION

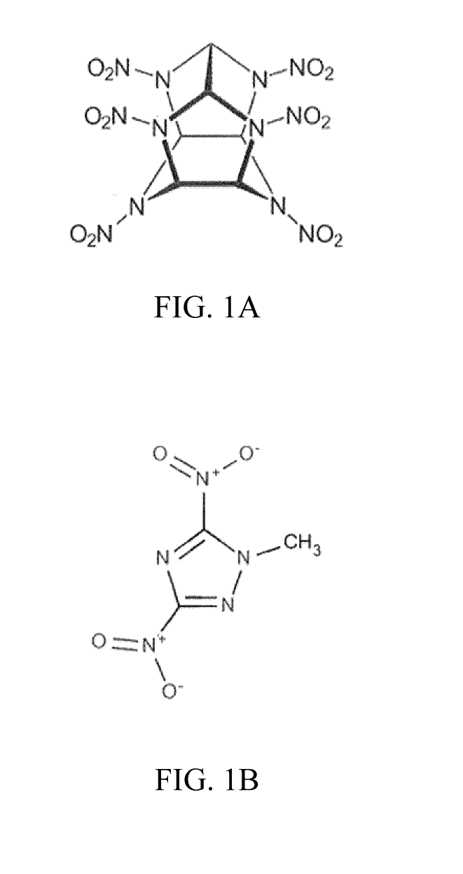

In some embodiments, the present disclosure is directed to a composite material including two energetic compounds. In some embodiments, the composite material is a cocrystal of those two energetic compounds. In some embodiments, the composite material includes a first energetic compound and a second energetic compound, wherein the composite material has a decreased sensitivity in comparison with a pure sample of the first energetic compound. In some embodiments, the first energetic compound is CL-20 (2,4,6,8,10,12-hexanitro-2,4,6,8,10,12-hexaazaisowurtzitane). The chemical structure of CL-20 is portrayed at FIG. 1A. In some embodiments, the second energetic compound is DNMT (1-methyl-3,5-dinitro-1,2,4-triazole).

DNMT, whose chemical structure is portrayed at FIG. 1B, was identified as a promising alternative given its performance and high density amongst the many candidates evaluated to replace TNT in field applications. It was thus surmised that this new energetic material might be a suitable coformer for the preparation of a novel CL-20-based cocrystal.

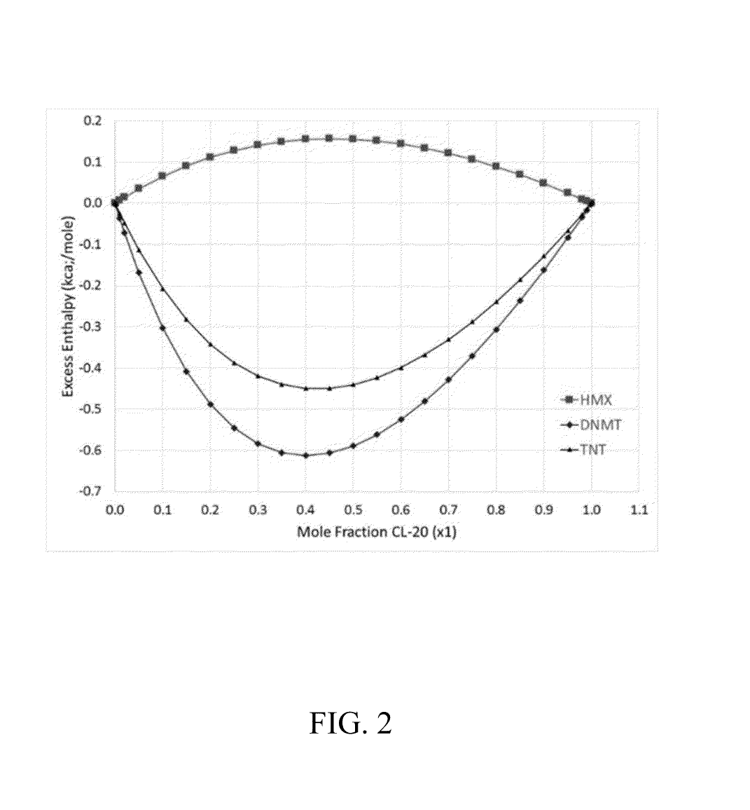

The feasibility of this cocrystal was first assessed by the comparing the excess enthalpy (Hₑₓ) of CL-20 with energetic coformers known from the literature. The heat of formation for the cocrystallization of CL-20 with octahydro-1,3,5,7-tetranitro-1,3,5,7-tetrazocine (HMX), TNT, and DNMT was thus calculated using COSMOtherm™, predictive property calculation software from COSMOlogic GmbH & Co. KG, Leverkusen, Germany. The comparison of the predicted Hₑₓ values offers a significant negative excess enthalpy at a mole fraction of CL-20 of 0.40, suggesting an increased stabilization offered by the cocrystallization of CL-20 and DNMT in a stoichiometric ratio (as seen in FIG. 2). The value calculated for this cocrystal pair compares favorably to other well-established coformers.

Several energetic compounds were screened for use as the second energetic compound which, when formed into a cocrystal with CL-20, resulted in a composite material with a pure sample of CL-20. As discussed above, it was discovered that almost all CL-20:X cocrystals resulted in a composite material significantly more sensitive than CL-20 itself. However, CL-20 cocrystals with DNMT surprisingly displayed the sought-after decreased sensitivity.

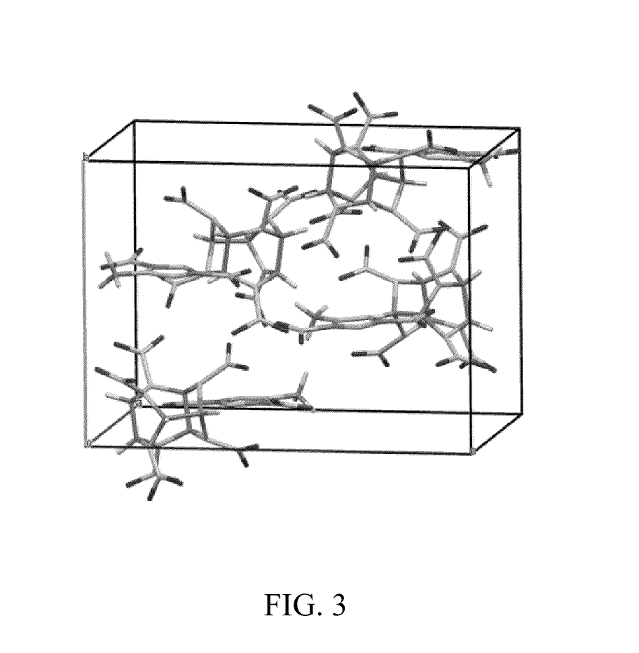

A colorless plate single crystal was eventually grown by the slow evaporation of a CL-20:DNMT solution. The crystal was found adequate for structural characterization by single-crystal X-ray diffraction. A nitro-π interaction between a nitro group of CL-20 and the triazole ring of DNMT, with distances ranging from 2.96 to 3.22 Å between the oxygen of the nitro function and the nitrogen of the triazole ring, was found. A distance of 2.60 Å between the oxygen and the hydrogen of the methyl group is at the limit of considerations for hydrogen bonding. As both components of the cocrystal are rich in nitro groups but lack hydrogen bond donors, the crystal packing is driven by electrostatic interactions generated by the polar nitro groups. The abundance of such anion-π interactions between adjacent molecules of both components results in significantly high density of the crystalline phase (p=1.883 g/cm³ at 95K). In some embodiments, the cocrystal structure features one molecule of CL-20 and one molecule of DNMT for a 1:1 stoichiometric/molar ratio. As shown in FIG. 3, in some embodiments, the unit cell is comprised of four molecules of CL-20 and four molecules of DNMT, where one molecule of CL-20 and one of DNMT molecule are symmetry independent.

The CL-20:DNMT cocrystal consistent with some embodiments of the present disclosure thus presents a density halfway between the ε-CL-20 polymorph (2.04 g/cm³) and DNMT (1.67 g/cm³). This value is on par with the reported density of 1.91 g/cm³ for β-HMX, one of the benchmarks for comparison of the potential of new entities for field applications. The molecular conformation of the CL-20 molecule in the cocrystal closely resembles that of the α polymorph, in which one of the nitro groups is in the “open” position. Conversely, in the thermodynamically stable form of CL-20 (ε-CL-20) two adjacent nitro groups are in the “open” position.

The conformational difference is a contributing factor to the lower density of the cocrystal as compared to the thermodynamically stable form of CL-20 as it creates small pockets of inaccessible space around every CL-20 molecule. These regions are evident from the unit cell packing diagram shown in FIG. 3. Interestingly, the density of the CL-20:DNMT cocrystal is reminiscent of the density of the α-form of CL-20 (1.92 g/cm³).

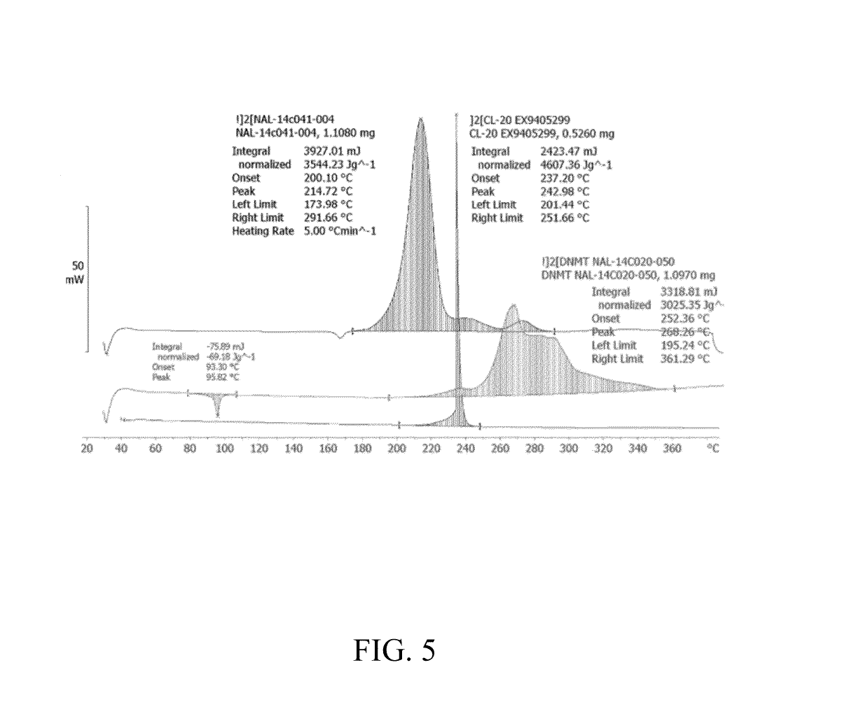

Analyzing CL-20:DNMT by differential scanning calorimetry (DSC) further confirmed that the desired cocrystal had been obtained and highlights characteristics of this new form of matter (as seen in FIG. 5). The DSC data shows the disappearance of DNMT's characteristic low temperature melt while presenting a new decomposition profile. The cocrystal onset temperature (200° C.) is lower than both discrete coformers and is preceded by a melt/phase transition starting at 165° C. Preliminary thermal assessment revealed that the cocrystal generates ca. 3544 Joules/gram, compared with 4607 and 3025 J/g for CL-20 and DNMT respectively. Interestingly, CL-20:DNMT seems to possess a greater energy on a mole basis with 2166 kJ/mol vs. 2019 kJ/mol for CL-20.

CL-20:DNMT was evaluated for sensitivity to stimuli using ERL impact, BAM friction and measured response to ESD. The cocrystal showed a sensitivity to impact similar to ε-CL20, its response to friction and ESD were found to be less pronounced.

TABLE 1
Sensitivity of the CL-20: DNMT cocrystal.

| | ERL Impact | | |
|---|---|---|---|
| Material | (50%, cm) | BAM Friction | ABL ESD |
| CL-20: DNMT | 25.5 | Go @ 128N; 10 | 20/20 No Goes |
| cocrystal | | No Goes @ 120N | @ 0.025 J |
| CL-20 | 26.2 | No Go @ 72N | 20/20 No Goes |
| | | | @ 0.051 J |
| DNMT | >100 | Go @ 288N; 10 | 20/20 No Goes |
| | | No Goes @ 252N | @ 0.25 J |

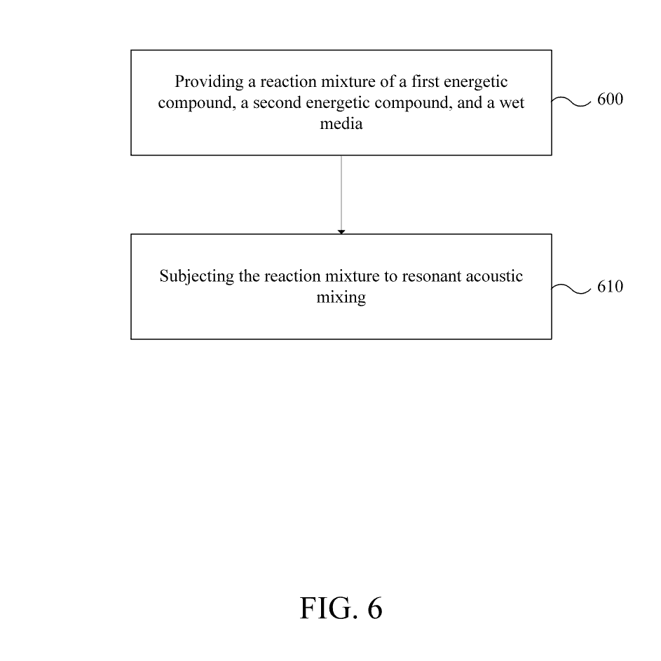

In some embodiments, the CL-20:DNMT cocrystal is produced through a process selected from the group consisting of: resonant acoustic mixing, solution crystallization, slurry crystallization, other mechanicochemical processes, and combinations thereof. In some embodiments, the CL-20:DNMT cocrystal is produced through a resonant acoustic mixing process. As shown in FIG. 6, in some embodiments, the resonant acoustic mixing process includes the steps of providing 600 a reaction mixture of a first energetic compound, a second energetic compound, and a wet media and subjecting 610 the reaction mixture to resonant acoustic mixing.

In some embodiments, the reaction mixture is subjected to resonant acoustic mixing for a period of about 50 minutes to about 1 hour, 10 minutes. In some embodiments, the reaction mixture is subjected to resonant acoustic mixing for a period of about 1 hour. In some embodiments, the reaction mixture is mixed at a g-force acceleration of about 70 g to about 90 g. In some embodiments, the reaction mixture is mixed at a g-force acceleration of about 80 g. Lower mixing forces result in a slower rate of cocrystal formation. Higher mixing forces increase the rate of cocrystal formation.

In some embodiments, the first energetic compound is CL-20 (2,4,6,8,10,12-hexanitro-2,4,6,8,10,12-hexaazaisowurtzitane). In some embodiments, the second energetic compound is DNMT (1-methyl-3,5-dinitro-1,2,4-triazole).

In some embodiments, the wet media is any suitable organic solvent. In some embodiments, the organic solvent is acetonitrile.

### Example

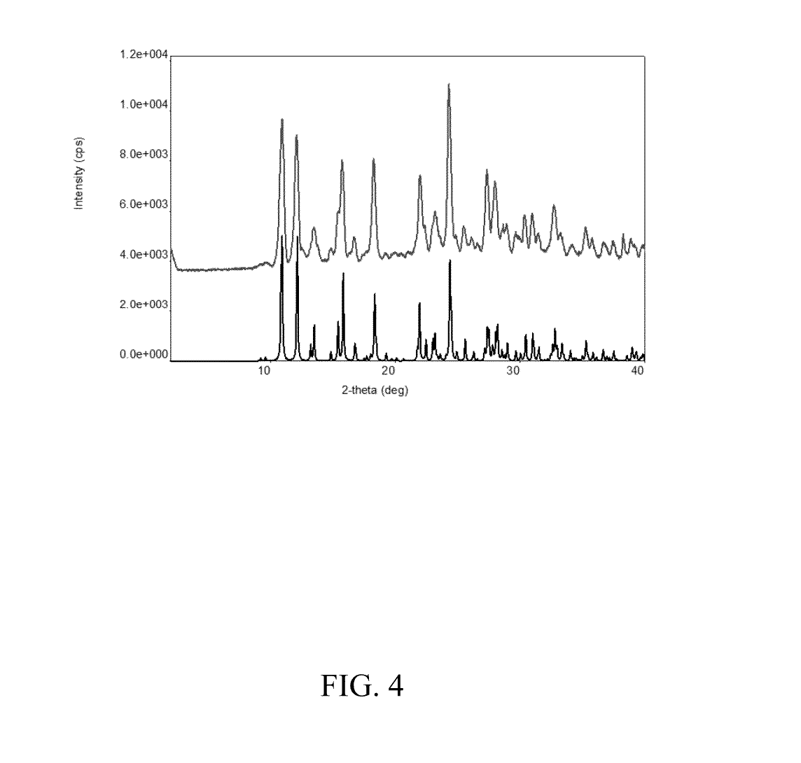

Conditions suitable for the production of the CL-20:DNMT cocrystals using RAM technology were screened. An evaluation of solvent additives and mixing times revealed that full conversion to the cocrystal could be achieved by mixing at 80-g force acceleration for 1 hour in the presence of acetonitrile as wet media. The resulting yellow powder recovered in quantitative yield was analyzed by powder X-ray diffraction (PXRD) to confirm that the cocrystal had indeed been formed. Comparing the reflections of the solids obtained by RAM with the PXRD pattern calculated from the single crystal structure confirmed the production of the desired CL-20:DNMT cocrystal (as seen in FIG. 4). Using this mechanicochemical technology, multi gram quantities of the novel CL-20:DNMT cocrystal were readily produced on the timescale of hours.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present disclosure.

What is claimed is:

1. A cocrystal comprising 2,4,6,8,10,12-hexanitro-2,4,6,8,10,12-hexaazaisowurtzitane and at least one additional energetic material, wherein the sensitivity of said cocrystal is less than the sensitivity of 2,4,6,8,10,12-hexanitro-2,4,6,8,10,12-hexaazaisowurtzitane.

2. The cocrystal according to claim 1, wherein said at least one additional energetic material is 1-methyl-3,5-dinitro-1,2,4-triazole.

3. The cocrystal according to claim 2, wherein a molar ratio of 2,4,6,8,10,12-hexanitro-2,4,6,8,10,12-hexaazaisowurtzitane to 1-methyl-3,5-dinitro-1,2,4-triazole is approximately 1:1.

4. The cocrystal according to claim 1 wherein said cocrystal is produced via a resonant acoustic mixing process.

5. The cocrystal according to claim 1 wherein said cocrystal is produced via a mechanicochemical process selected from the group consisting of: solution crystallization, slurry crystallization, and the like.

6. The cocrystal according to claim 2 wherein a unit cell of said cocrystal comprises about 4 molecules of 2,4,6,8,10,12-hexanitro-2,4,6,8,10,12-hexaazaisowurtzitane and about 4 molecules of 1-methyl-3,5-dinitro-1,2,4-triazole.

7. The cocrystal according to claim 2 wherein said cocrystal has a crystalline phase density at 95 Kelvin of about 1.8 g/cm³ to about 1.9 g/cm³.

8. A method of making a composite material including 2,4,6,8,10,12-hexanitro-2,4,6,8,10,12-hexaazaisowurtzitane and 1-methyl-3,5-dinitro-1,2,4-triazole comprising the following steps:
providing a reaction mixture of 2,4,6,8,10,12-hexanitro-2,4,6,8,10,12-hexaazaisowurtzitane, 1-methyl-3,5-dinitro-1,2,4-triazole, and a wet media; and
subjecting said reaction mixture to resonant acoustic mixing.

9. The method of making a composite material according to claim 8, wherein said reaction mixture is subjected to resonant acoustic mixing for a period of at least about 1 hour.

10. The method of making a composite material according to claim 8, wherein said reaction mixture is mixed at a g-force acceleration of about 80 g.

11. The method of making a composite material according to claim 8, wherein said wet media is an organic solvent.

12. The method of making a composite material according to claim 11, where said organic solvent is acetonitrile.

13. A composite material including 2,4,6,8,10,12-hexanitro-2,4,6,8,10,12-hexaazaisowurtzitane and 1-methyl-3,5-dinitro-1,2,4-triazole having a 1:1 molar ratio of 2,4,6,8,10,12-hexanitro-2,4,6,8,10,12-hexaazaisowurtzitane to 1-methyl-3,5-dinitro-1,2,4-triazole.

14. The composite material according to claim 13 wherein said composite material is produced via a resonant acoustic mixing process.

15. The composite material according to claim 13 wherein said composite material is a cocrystal.

16. An energetic formulation including the composite material according to claim 13.
